(19) United States
(12) Patent Application Publication
(10) Pub. No.: US 2023/0310686 A1
(43) Pub. Date: Oct. 5, 2023

(54) MULTI-FUNCTIONAL POWDER IMCREE AND MANUFACTURING METHOD THEREFOR

(71) Applicant: YCIMCREE CO., LTD., Seoul (KR)

(72) Inventor: Woo Yong BYON, Seoul (KR)

(73) Assignee: YCIMCREE CO., LTD., Seoul (KR)

(21) Appl. No.: 18/013,399

(22) PCT Filed: Sep. 14, 2021
(86) PCT No.: PCT/KR2021/012449

§ 371 Date: Dec. 28, 2022

(30) Foreign Application Priority Data
Sep. 22, 2020 (KR) 10-2020-0122156

(51) Int. Cl. A61L 9/18; A61L 9/014

(52) U.S. Cl. CPC A61L 9/014; A61L 9/18; A61L 2101/12

(57) ABSTRACT

Presented here is a multi-functional powder which: comprises the first component that includes one selection from the group consisting of silicon oxide (SiO2), aluminum oxide (Al2O3), calcium oxide (CaO), magnesium oxide (MgO), and combinations thereof; and the second component that includes at least one selection from the group consisting of iron oxide (Fe2O3), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof; whose far-infrared emission rate for the wavelength region of 5 µms to 20 µms at 37° C. is between 0.5% and 5%; whose harmful gas deodorization balance index under the following Formula 1 is between 0.8 and 2.0; and whose bacterial reduction rate (%) is 90% or greater for each of staphylococcus aureus (ATCC 6538), Escherichia coli (ATCC 8739), Klebsiella pneumonia (ATCC 4352), and Pseudomonas aeruginosa (ATCC 10145).

### TECHNICAL FIELD

This relates to a multi-functional powder that can be utilized as an eco-friendly material in various technical fields such as the construction, the agricultural and fishery industry, and the automobile industry as well as its manufacturing method.

### BACKGROUND TECHNOLOGY

Throughout the history, humanity has been evolving itself while striving to invent better and fitter instruments under the changing environment. In this process, various materials have been developed, and in the modern industrial society, the development of new materials is highly critical to a nation’s major industries, such as electrical and electronic, medical, automobile, textile, architecture, to name a few. New materials can be classified into metallic, inorganic non-metallic, new polymeric, composite materials, and so forth, according to the quality of materials. Inorganic non-metallic materials are made by processing natural or artificially synthesized inorganic compounds, which can be utilized as environmentally friendly materials if developed into novel materials by properly processing their natural ingredients. As we enter the era of the 4th industrial revolution, the impact of big data and artificial intelligence are boosting up economic productivity and human quality of life. In terms of population structure, aging of the population has started and accordingly, the intensified pursuit of health and safety is a modern trend and the technology for bio-based materials and regenerative medical material technology are taking the spotlight. With respect to climate and environment, eco-friendly industries and clean energy technology are expanding their clout, and accordingly, technologies for materials that enable eco-friendly energy as well as resource recycling are emerging as technologies for the future. Considering the needs of the times, it is imperative to develop environmentally friendly novel materials that may reduce negative factors such as environmental pollution and harmful chemicals and promote human health and welfare.

### DETAILS OF THE INVENTION

### Technical Tasks

One embodiment of this Invention is to provide a multi-functional powder, which emits far-infrared radiation to carry out a warming action maintaining the body temperature at an optimal level and a hydration/dehydration action keeping the human body at an appropriate level, and promotes metabolism while enhancing immunity through the balance of nutrients. Furthermore, the multi-functional powder realizes the effect of deodorizing harmful gas intended toward excellent antibacterial action, particularly mitigating the odor from ammonia gas and formaldehyde gas, which cause irritation and harmful effect to bio-tissues.

Another embodiment of this Invention, manufactured in various forms and having a wide range of utility, provides storage devices utilizing the multi-functional powder that provides excellent capability to store organic materials for prolonged period.

### Solutions to the Tasks

One embodiment of this Invention provides a multi-functional powder which: comprises the first component that includes one selection from the group consisting of silicon oxide (SiO2), aluminum oxide (Al2O3), calcium oxide (CaO), magnesium oxide (MgO), and combinations thereof; comprises the second component that includes at least one selection from the group consisting of iron oxide (Fe2O3), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof; and whose far-infrared emission rate for the wavelength region of 5 µms to 20 µms at 37° C. is between 0.5% and 5%, and whose harmful gas deodorization balance index under the following Formula 1 is between 0.8 and 2.0.

$$\left\{\left(\text{Cai - Caf}\right) / \left(\text{Cai}\right)\right\} / \left\{\left(\text{Cfi - Cff}\right) / \left(\text{Cfi}\right)\right\} \qquad \text{[Formula 1]}$$

In Formula 1 above: Cai is the initial gas concentration value after injecting 100 ppm volume % of ammonia gas into a tightly sealed space with the volume of 2 L under the temperature of 23±5° C. and a relative humidity of 45±10%; Caf is the ammonia gas concentration value after 120 minutes’ passage from placing 1.0 g of the multi-functional powder in the aforesaid tightly sealed space; Cfi is the initial gas concentration value after injecting 100 ppm volume% of formaldehyde gas into a tightly sealed space with the volume of 2 L under the temperature of 23±5° C. and a relative humidity of 45±10%; and Cff is the formaldehyde gas concentration value after 120 minutes’ passage from placing 1.0 g of the multi-functional powder in the aforesaid tightly sealed space.

The aforesaid multi-functional powder may have an ammonia deodorization rate of 90% or greater, according to Formula 2 below.

$$\text{Ammonia deodorization rate} \left(\%\right) = \left(\text{Cai - Caf}\right) / \text{Cai} \text{X 100} \qquad \text{[Formula 2]}$$

In Formula 2, aforesaid Cai is the initial gas concentration value after injecting 100 ppm volume % of gas into a tightly sealed space with the volume of 2 L under the temperature of 23° C. and a relative humidity of 45%; and aforesaid Caf is the gas concentration value after 120 minutes’ passage from placing 1.0 g of the multi-functional powder in the aforesaid tightly sealed space.

The aforesaid multi-functional powder may have an ammonia deodorization rate of 50% or greater, according to Formula 3 below

$$\text{Formaldehyde deodorization rate} \left(\%\right) = \left(\text{Cfi} - \text{Cff}\right) / \text{Cfi} \text{X 100} \qquad \text{[Formula 3]}$$

In Formula 3, aforesaid Cfi is the initial gas concentration value after injecting 100 ppm volume % of gas into a tightly sealed space with the volume of 2 L under the temperature of 23° C. and a relative humidity of 45%; and aforesaid Cff is the gas concentration value after 120 minutes’ passage from placing 1.0 g of the multi-functional powder in the aforesaid tightly sealed space.

The aforesaid multi-functional powder may have the bacterial reduction rate (%) of 90% or greater in accordance with Formula 4 for each of staphylococcus aureus (ATCC 6538), Escherichia coli (ATCC 8739), Klebsiella pneumonia (ATCC 4352), and Pseudomonas aeruginosa (ATCC 10145).

$$\text{Bacterial reduction rate} \left(\%\right) = \left(\text{X-Y}\right) / \text{X} \text{*100} \qquad \text{[Formula 4]}$$

In Formula 4, aforesaid X is the number of colony-forming unit (CFU) in the controlled group, and Y is the number of CFU in the experimental group treated with the multi-functional powder.

The weight ratio of the first component to the second component of the multi-functional powder should be between 70:30 and 100:0.

According to an embodiment, the multi-functional powder comprises the first component that includes calcium oxide (CaO) and magnesium oxide (MgO) and the second component that includes sulfur trioxide (S3O) and strontium oxide (SrO), and the weight ratio of the first component to the second component may be 95:5 or greater and less than 100:0.

According to another embodiment, the multi-functional powder comprises the first component that includes silicon oxide (SiO2), aluminum oxide (Al2O3) and calcium oxide (CaO), and the second component that includes iron oxide (Fe2O3), and the weight ratio of the first component to the second component may be 75:25 or greater and less than 100:0.

Another embodiment of this Invention provides a multi-functional storage devices that contain functional material that comprises the multi-functional powder or a processed product thereof and a base material containing a resin or clay, as well as multi-functional storage devices that include a storage target receiving part and a storage target protecting part.

### Effects of the Invention

The multi-functional powder may cause a beneficial effect to the human body based on far-infrared radiation feature. Specifically, the far-infrared rays by the multi-functional powder carries out a warming action maintaining the body temperature at an optimal level and a hydration/dehydration action keeping the human body at an appropriate level, and promotes metabolism while enhancing immunity through the balance of nutrients. In addition, it helps the expansion of capillaries and the cell tissue formation thereby promoting blood circulation and generating an effect of perspiration and pain relief, as well as enhanced metabolism.

Moreover, the multi-functional powder delivers an effect of deodorizing harmful gas, and in particular, has an excellent advantage of mitigating the odor from gases that are irritating and harmful to bio-tissues, such as ammonia and formaldehyde.

### Implementation Mode of the Invention

Advantages and features of this Invention, and a method of achieving them will become clear by referring to the following examples. However, this Invention is not limited to the embodiments disclosed below, but will be implemented in diversified forms. The embodiments presented here are intended to complete the disclosure of this Invention and to fully inform those with ordinary knowledge in the technology field of the scope of this Invention. This Invention is only defined by the classifications in the claims.

In the drawings, the thickness is presented enlarged in order to clearly express the various strata and areas. Also, the drawings show the thickness of some strata and areas in an exaggerated manner, for convenience of explanation. Throughout the entire Specification, the same reference numerals and codes refer to the same elements.

In this Specification, when a part such as stratum, membrane, area, layer, etc. is said to be “above” or “upper to” another part, this is not limited to the part placed immediately on that another part, but encompasses the case where some intermediate parts exist in the middle. Conversely, when the text mentions that a part is “immediately above” another part, it means that there is no intermediate part embedded in the middle. As a corollary, when a part such as stratum, membrane, area, layer, etc. is said to be “under” or “below” another part, this is not limited to the part placed immediately under that another part, but encompasses the case where some intermediate parts exist in the middle. As a corollary, when a part is said to be “immediately under” another part, it means that there is no intermediate part in the middle.

- [34] One embodiment of this Invention provides a multi-functional powder which: comprises the first component that includes one selection from the group consisting of silicon oxide (SiO2), aluminum oxide (Al2O3), calcium oxide (CaO), magnesium oxide (MgO), and combinations thereof and the second component that includes at least one selection from the group consisting of iron oxide (Fe2O3), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof; and whose far-infrared emission rate for the wavelength region of 5 µms to 20 µms at 37° C. is between 0.5% and 5%, and whose harmful gas deodorization balance index under the following Formula 1 is between 0.8 and 2.0.

$$\left\{\left(\text{Cai - Caf}\right) / \text{Cai}\right\} / \left\{\left(\text{Cfi - Cff}\right) / \text{Cfi}\right\} \qquad \text{[Formula 1]}$$

In Formula 1 above: Cai is the initial gas concentration value after injecting 100 ppm volume % of ammonia gas into a tightly sealed space with the volume of 2 L under the temperature of 23±5° C. and a relative humidity of 45±10%; Caf is the ammonia gas concentration value after 120 minutes’ passage from placing 1.0 g of the multi-functional powder in the aforesaid tightly sealed space; Cfi is the initial gas concentration value after injecting 100 ppm volume% of formaldehyde gas into a tightly sealed space with the volume of 2 L under the temperature of 23±5° C. and a relative humidity of 45±10%; and Cff is the formaldehyde gas concentration value after 120 minutes’ passage from placing 1.0 g of the multi-functional powder in the aforesaid tightly sealed space.

Various minerals extracted from natural materials have high potential as environmentally friendly components. In addition, the features and effects that can be implemented depending on their resource materials, final composition, and manufacturing method can be adjusted in a wide range. According to an embodiment, the multi-functional powder has the advantage of implementing a far-infrared radiation function and a harmful gas deodorization function on a concurrent basis.

* The aforesaid multi-functional powder’s far-infrared radiation rate in a wavelength band between 5 µms and 20 µms at 37° C. may be between 0.5% and 5%, that is: between 0.5% and 4%, for example; or between 0.5% and 3.5%, for example; or between 0.5% and 2%, for example; or between 0.5% and 1.5%, for example (All the percentage figures are approximates). The “far-infrared radiation rate” means the radiation rate for at least one wavelength band belonging to the wavelength bands between 5 µms to 20 µms.

The multi-functional powder’s far-infrared radiation energy in a wavelength region of 5 µms to 20 µms at 37° C. may be 1.00 × 102 W/m²•µm or higher, that is: between 1.00 × 102 W/m²•µm and 5.00 × 102 W/m²•µm, for example; or between 2.00× 102 W/m²•µm and 4.00×102 W/m²•µm, for example (All the W/m²•µm figures are approximates.). The aforesaid far-infrared radiation energy is defined as the sum of energy emitted in the aforesaid wavelength band, and can be obtained by totaling the following areas of the radiation energy graph by wavelength.

The maximum peak value of the far-infrared radiation energy of the multi-functional powder may appear in the band between approximately 6 µm and 12 µm at 37° C. In this connection, the maximum peak value of the far-infrared radiation energy may be between approximately 25 W/m2•µm and 40 W/m2•µm.

The aforesaid multi-functional powder has the aforesaid far-infrared radiation particulars under a 37° C., a condition similar to the body temperature. That is, the far-infrared radiation from the multi-functional powder carries out a warming action maintaining the body temperature at an optimal level and a hydration/dehydration action keeping the human body at an appropriate level, and promotes metabolism while enhancing immunity through the balance of nutrients. Furthermore, it helps the expansion of capillaries and the cell tissue formation thereby promoting blood circulation and generating an effect of perspiration and pain relief, as well as enhanced metabolism. For example, the aforesaid far-infrared radiation rate and radiant energy can be measured by KIFA-FI -1005 standard specification method.

The aforesaid multi-functional powder has excellent deodorizing function for each of ammonia gas and formaldehyde gas, realizing the effect of concurrently deodorizing the two gases to a degree higher than certain predesignated level, a generally difficult task to carry out. The harmful gas deodorization balance index in Formula 1) shows the degree of balance between deodorization performance for ammonia gas and that for formaldehyde gas. The harmful gas deodorization balance index according to Formula 1 may be between 0.8 and 2.0, that is: between 0.8 (inclusive) and 2.0 (not inclusive), for example; or between 0.8 and 1.7, for example; or between 0.9 and 1.6 (Figures are approximates except for an identifier.) The aforesaid multi-functional powder’s deodorization performance is not extremely skewed to either of the two harmful gases, ammonia and formaldehyde, but shows a balanced deodorization performance corresponding to the harmful gas deodorization balance index for both harmful gases. In particular, as it implements a deodorization balance corresponding to the harmful gas deodorization balance index in the aforesaid range, the multi-functional powder can effectively carry out gas deodorization performance under conditions in which both ammonia and formaldehyde exist.

In one embodiment, the multi-functional powder may have an ammonia deodorization rate (%) of 90% or greater according to Formula 2 below.

$$\text{Ammonia deodorization rate} \left(\%\right) = \left(\text{Cai - Caf}\right) \text{X 100} \qquad \text{[Formula 2]}$$

In Formula 2, aforesaid Cai is the initial gas concentration value after injecting 100 ppm volume % of gas into a tightly sealed space with the volume of 2 L under the temperature of 23° C. and a relative humidity of 45%; and aforesaid Caf is the gas concentration value after 120 minutes’ passage from placing 1.0 g of the multi-functional powder in the aforesaid tightly sealed space.

The multi-functional powder’s ammonia deodorization rate (%) according to Formula 2 may be 90% or greater, that is: 92% or greater, for example, or 94% or greater, for example, or 95% or greater, for example. (All the percentage figures are approximates.) Ammonia is a harmful gas that can damage skin tissue when it contacts the human body and can cause various inflammations such as laryngitis and bronchitis. By virtue of the fact that it has first component and the second component as well as a particle shape of a specific structure and size, the multi-functional powder can carry out such an excellent ammonia gas removal action.

In one embodiment, the multi-functional powder may have a formaldehyde deodorization rate (%) of 50% or greater according to Formula 3 below.

$$\text{Formaldehyde deodorization rate} \left(\%\right) = \left(\text{Cfi - Cff}\right) / \text{Cti} \text{X 100} \qquad \text{[Formula 3]}$$

In Formula 3, aforesaid Cfi is the initial gas concentration value after injecting 100 ppm volume % of gas into a tightly sealed space with the volume of 2 L under the temperature of 23° C. and a relative humidity of 45%; and aforesaid Cff is the gas concentration value after 120 minutes’ passage from placing 1.0 g of the multi-functional powder in the aforesaid tightly sealed space.

The multi-functional powder’s formaldehyde deodorization rate (%) may be 50% or greater according to Formula 3, that is: 55% or greater, for example; or 60% or greater, for example; or 90% or greater, for example; or 95% or greater, for example (All the percentage figures are approximate). Flammable and colorless, formaldehyde is a harmful gas with a low ignition point and a high risk of explosion, causing chronic irritation to the respiratory tract of the subject who is repeatedly exposed to it. By virtue of having the first and second component as aforesaid as well as a particle shape of a certain structure and size, it is capable of implementing such excellent performance to remove formaldehyde gas.

In one embodiment, the multi-functional powder may have the bacterial reduction rate (%) of 90% or greater in accordance with Formula 4 for each of staphylococcus aureus (ATCC 6538), Escherichia coli (ATCC 8739), Klebsiella pneumonia (ATCC 4352), and Pseudomonas aeruginosa (ATCC 10145).

$$\text{Bacterial reduction rate} \left(\%\right) = \left(\text{X-Y}\right) / \text{X} \text{*100} \qquad \text{[Formula 4]}$$

In Formula 4, aforesaid X is the number of colony-forming unit (CFU) in the controlled group, and Y is the number of CFU in the experimental group treated with the multi-functional powder.

For example, with respect to each of staphylococcus aureus (ATCC 6538), Escherichia coli (ATCC 8739), Klebsiella pneumonia (ATCC 4352), and Pseudomonas aeruginosa (ATCC 10145), the aforesaid bacterial reduction rate (%) may be 90% or greater, that is: 95% or greater, for example; or between 96% and 100%, for example; or between 97% and 100%, for example; or between 99% and 100%, for example (All of the percentage figures are approximates.) By implementing the aforesaid range of bacterial reduction, the aforesaid multi-functional powder has an advantage of being used for applied fields where antibacterial function is required, and can greatly enhance its functions friendly to human body and environment.

Aforesaid multi-functional powder comprises the first component that includes one selection from the group consisting of silicon oxide (SiO2), aluminum oxide (Al2O3), calcium oxide (CaO), magnesium oxide (MgO), and combinations thereof; and the second component that includes at least one selection from the group consisting of iron oxide (Fe2O3), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof.

The first component of the aforesaid multi-functional powder may further include a different component in addition to its specific categories indicated above, and the second component may also include a different component in addition to its specific categories indicated above.

The weight ratio of the first component and the second component of the multi-functional powder may be between 70:30 (inclusive) and 100:0 (not inclusive), that is: between 70:30 and 99.9:0.1, for example; or between 75:25 (inclusive) and 100:0 (not inclusive), for example; or between 75:25 and 99.9:0.1, for example; or between 80:20 and 99.9:0.1, for example; or between 85:15 and 99.9:0.1, for example; or between 90:10 and 99.9:0.1, for example; or between 75:25 and 95:5, for example; or between 75:25 and 90:10, for example; or between 75:25 and 89:11, for example; or between 80:20 and 89:11, for example; or between 96:4 and 99.9:0.1, for example; or between 97:3 and 99.9:0.1 (All the figures are approximates). By virtue of the fact that the weight ratio of the first component to the second component satisfies the aforesaid range, the far-infrared radiation function and the harmful gas deodorization function may be realized at the target level on a concurrent basis.

According to an embodiment, the aforesaid multi-functional powder (hereinafter, it may be referred to as “multi-functional powder I”) comprises the first component that includes calcium oxide (CaO) and magnesium oxide (MgO) and the second component that includes sulfur trioxide (S3O) and strontium oxide (SrO), and the weight ratio of the first component to the second component may be 95:5 or greater and less than 100:0.

The first component may include a relatively small amount of different components in addition to calcium oxide (CaO) and magnesium oxide (MgO), and the second component may also include a relatively small amount of different components in addition to sulfur trioxide (S3O) and strontium oxide (SrO). In this case, ‘relatively small amount of different component’ means a different component having a lower content than specified for each component, such as calcium oxide (CaO), magnesium oxide (MgO), sulfur trioxide (S3O), and strontium oxide (SrO).

Specifically, the weight ratio of the first component and the second component of the multi-functional powder | may be between 96:4 and 99.1, that is: between 97:3 and 99:1, for example (All the figures are approximates). By virtue of the fact that the weight ratio of the first component to the second component satisfies such range, the far-infrared radiation function and the harmful gas deodorization function may be realized at the target level. Especially, it has excellent anti-bacterial function and deodorization function, imparting an outstanding advantage of human-friendly features when compared to artificial chemical substances.

Specifically, in the multi-functional powder |, the first component includes calcium oxide (CaO) and magnesium oxide (MgO), and of the total 100 weight% of the first component, calcium oxide (CaO) may account for 90 weight% or greater. For example, of the total 100 weight% of the first component, the calcium oxide (CaO) may be 91 weight% or greater, or 92 weight% or greater, for example; or 93 weight% or greater and 98 weight% or less (All the weight % figures are approximates.). As such, when the main component of the first component is calcium oxide (CaO), it may be more advantageous when antibacterial and deodorizing performance are taken into account.

Specifically, in the multi-functional powder |, the first component may include calcium oxide (CaO), magnesium oxide (MgO), and aluminum oxide (Al2O3). In this case, of the total 100 weight% of the first component, calcium oxide (CaO) may be 90 weight% or greater, and the content of the magnesium oxide (MgO) may be greater than the content of the aluminum oxide (Al2O3). More specifically, the weight ratio of the magnesium oxide (MgO) to the aluminum oxide (Al2O3) may be between 5:1 and 25:1, that is: between 8:1 and 24:1, for example; or between 10:1 and 25:1, for example; and between 15:1 and 20:1, for example (Figures are approximates except for “100”.). By satisfying these conditions, the composition of the first component may be more advantageous when antibacterial and deodorizing performance are taken into account.

Specifically, in the multi-functional powder |, the second component may include sulfur trioxide (S3O) and strontium oxide (SrO), and, in the second component, the content of the sulfur trioxide (S3O) may be greater than the content of the strontium oxide (SrO). More specifically, the weight ratio of the sulfur trioxide (S3O) to the strontium oxide (SrO) may be between 1:1 (not inclusive) and 10:1 (inclusive), that is: between 2:1 and 8:1, for example; or between 2:1 and 6:1, for example; or between 3:1 and 6:1, for example (Figures are approximates.). By satisfying these conditions, the composition of the second component may be more advantageous when antibacterial and deodorizing performance are taken into account.

According to another embodiment, the aforesaid multi-functional powder (hereinafter, it may be referred to as “multi-functional powder ll”) comprises the first component that includes silicon oxide (SiO2), aluminum oxide (Al2O3) and calcium oxide (CaO) and the second component that includes iron oxide (Fe2O3), and the weight ratio of the first component to the second component may be 75:25 or greater and less than 100:0 (Figures are approximates.).

The first component may include a relatively small amount of different components in addition to silicon oxide (SiO2), aluminum oxide (Al2O3) and calcium oxide (CaO), and the second component may also include a relatively small amount of different components in addition to iron oxide (Fe2O3). In this case, ‘relatively small amount of different component’ means a different component having a lower content than specified for each component, such as silicon oxide (SiO2), aluminum oxide (Al2O3), calcium oxide (CaO)and iron oxide (Fe2O3).

Specifically, the weight ratio of the first component and the second component of the multi-functional powder II according to another embodiment may be between 75:25 and 99:1, that is: between 85:15 and 99:1, for example; between 90:10 and 99:1, for example; and between 95.5 and 99.1, for example (All the figures are approximates). By virtue of the fact that the weight ratio of the first component to the second component satisfies such range, the far-infrared radiation function and the harmful gas deodorization function may be realized at the target level.

Specifically, in the multi-functional powder II, the first component includes silicon oxide (SiO2), aluminum oxide (Al2O3) and calcium oxide (CaO), and of the total 100 weight% of the first component, the silicon oxide (SiO2) and aluminum oxide (Al2O3) may account for 80 weight% or greater. For example, of the total 100 weight% of the first component, the silicon oxide (SiO2) and aluminum oxide (Al2O3) may be 85 weight% or greater, that is: between 85 weight% and 99 weight%, for example; or between 85 weight% and 99 weight%, for example. (All the weight % figures are approximates.). As such, when the main component of the first component is silicon oxide (SiO2) and aluminum oxide (Al2O3), its advantage of excellent infra-red radiation performance can be maximized.

Specifically, in the multi-functional powder II, the first component may have silicon oxide (SiO2) in equal to or greater content than aluminum oxide (Al2O3). More specifically, the weight ratio of the silicon oxide (SiO2) to aluminum oxide (Al2O3) may be between 1:1 and 4:1, that is: or between 1:1 and 3:1, for example; or between 1:1 and 2:1, for example; or between 1:1 and 1.5:1, for example; or between 1:1 and 1.2:1, for example (Figures are approximates.). By satisfying these conditions, the aforesaid multi-functional powder may implement excellent antibacterial and deodorizing performance.

In one embodiment, the second component of the multi-functional powder II may include iron oxide (Fe2O3) and sodium oxide (Na2O). In this case, the content of the iron oxide (Fe2O3) may be equal to or greater than the content of sodium oxide (Na2O). As a result, it is capable of implementing more improved far-infrared radiation action. Specifically, the weight ratio of the iron oxide (Fe2O3) to the sodium oxide (Na2O) may be between 1:1 and 3:1, that is: between 1:1 and 2:1, for example; or between 1:1 and 1.8:1, for example; or between 1.1:1 and 1.5:1, for example (Figures are approximates.). As such, the aforesaid multi-functional powder may be advantageous in implementing excellent far-infrared radiation performance and deodorization performance on a concurrent basis.

The composition of the aforesaid multi-functional powder can be identified by using an X-Ray Fluorescence (XRF), and can be controlled by raw materials as well as various processing conditions during the course of producing the aforesaid multi-functional powder.

The oxidation-reduction potential (ORP) of the aforesaid multi-functional powder may be 600 mV or less, for example, that is: the ORP of the aforesaid multi-functional powder may be between -20 mV and 600 mV, for example; or between -20 mV and 550 mv, for example; or between -20 mv and 250 mv, for example; or between -10 mV and 220 mV, for example; or between -10 mV and 10 mV, for example; or between 170 mV and 200 mV, for example (All of the mV figures are approximates.). The aforesaid ORP was obtained by a process that 1 g of the multi-functional powder is dispersed in 10 mL of deionized water, which undergoes 24-hour extracting from the shake, and measurement is taken from the supernatant formed by centrifuging said extract from the shake. The aforesaid multi-functional powder, having a power of reduction corresponding to aforesaid range of the OPR, can implement a function that is friendly to the human body and the environment and can be more advantageous in deodorizing and antibacterial action.

The hydrogen ion concentration (pH) of the aforesaid multi-purpose powder may be 5.0 and 13.5, that is: between 5.0 and 7.0, for example; or between 5.5 and 7.0, for example; or between 7.0 and 13.5, for example; or between 8.0 and 13.5, for example; or between 10.0 and 13.0, for example (Figures are approximates). In this case, the hydrogen ion concentration (pH) was obtained by measuring the pH of the supernatant formed on the liquid solution of 10 mL of distilled water having 1.0 g of aforesaid multi-functional powder dispersed therein, which was placed at a temperature of 24±2° C. and a relative humidity of 35±5% for 30 minutes. The aforesaid multi-functional powder, having a pH in the aforesaid range, can implement a function that is friendly to the human body and the environment and can be more advantageous in deodorizing and antibacterial action.

The cation exchange capacity (CEC) of the aforesaid multi-functional powder may be between 5 cmol(+)/kg and 50 cmol(+)/kg, that is, between 5 cmol(+)/kg and 45 cmol(+)/kg, for example; or between 10 cmol(+)/kg and 45 cmol(+)/kg, for example; or between 15 cmol(+)/kg and 45 cmol(+)/kg, for example; or between 15 cmol(+)/kg and 25 cmol(+)/kg, for example; or between 30 cmol(+)/kg and 45 cmol(+)/kg, for example (All the cmol(+)/kg figures are approximates.). The aforesaid cation exchange capacity refers to the positive ion substitution capacity and specifically to the total amount of exchangeable cation possessed by the aforesaid multi-functional powder, which can be utilized as an indicator of capacity to hold nutrients. Since the aforesaid multi-functional powder possesses a capability of cation substitution corresponding to the aforesaid range of cation exchange capacity, it may exert excellent deodorizing performance and harmful metal adsorption performance, and may be advantageous in implementing a function to purify wastewater, among others.

The electrical conductivity (EC) of the multi-functional powder may be 0.01 ds/m(1:10) or greater, that is: between 0.01 ds/m(1:10) and 10 ds/m(1:10), for example; or between 0.01 ds/m(1:10) and 2 ds/m(1:10), for example; or between 5 ds/m(1:10) and 10 ds/m(1:10), for example. Inasmuch as aforesaid EC satisfies the aforesaid range, the aforesaid multi-functional powder can maximize the friendliness to human body and environment (All of the ds/m figures are approximates.].

The multi-functional powder may have an average particle diameter (D50) between 20 µm and 200 µm, that is: between 20 µm and 150 µm, for example; or between 20 µm and 100 µm, for example; or between 20 µm and 80 µm, for example; or between 20 µm and 60 µm, for example; or between 25 µm and 55 µm, for example; or between 30 µm and 50 µm, for example (All the µm figures are approximates.). By using a multi-functional powder having a size of aforesaid range, it gives advantage in transportation and subsequent processing, and it may be advantageous in achieving excellent far-infrared radiation performance and deodorization performance.

Another embodiment of this Invention provides multi-functional storage devices that contain a functional material that comprises the multi-functional powder or a processed product thereof and a base material containing a resin or clay, as well as multi-functional storage devices that include a storage target receiving part and a storage target protecting part.

In regard to the multi-functional storage devices, all matters relating to the multi-functional powder are as described above.

As a material that implements various functions such as far-infrared radiation performance and ammonia deodorization performance derived from the multi-functional powder, the aforesaid functional material may include the multi-functional powder itself or a product processed from the multi-functional powder. The aforesaid product processed from multi-functional powder is a material to which processing has been made such as heat treatment, surface treatment, or phase conversion without compromising the original function of the powder. The functional material may include the aforesaid multi-functional powder, a processed product therefrom, or a mixture of both.

As a mixture with the functional material, the base material constitutes a skeleton for the multi-functional goods and may include resin or clay.

When the base material includes a resin, the resin may include a selection from a group consisting of the components including but not limited to polyethylene (PE), polypropylene (PP), polyvinylchloride (PVC), polystyrene, (PS), polyacrylonitrile (PAN), acrylonitrile butadiene styrene (ABS), styrene-acrylonitrile copolymer, polymethyl methacrylate (PMMA), polyethylene terephthalate (PET), polyamide (PA), polycarbonate (PC), polyphenylene sulfide (PPS), phenolics, epoxy resin, polyurethane, and combinations thereof.

When the base material includes clay, the clay may include five-colored fortune soil.

In one embodiment, the aforesaid multifunctional storage devices may include aforesaid functional materials between 1 and 80 weight-multiples to aforesaid 100 weight-multiple, that is: between 1 and 40 weight-multiples, for example; or between 1 and 20 weight-multiples, for example (Figures are approximates except for “100”.).

The aforesaid multifunctional storage devices can be produced in the process that may include: a stage to prepare a mixed raw material in which the aforesaid functional material and the aforesaid base material are mixed; and a stage of molding aforesaid mixed raw materials to produce goods equipped with the storage target receiving part and the storage target protecting part.

In one embodiment, when the base material includes resin, the aforesaid molding of the mixed raw materials may include the stage to melt the mixed raw material at a temperature equal to or higher than the melting point of said resin and then process pressing-out, injection, or coating.

In another embodiment where the base material includes clay, the molding of the aforesaid mixed raw material may include a stage of formulating the mixed raw material at a temperature between 1,000° C. and 1,500° C.

The aforesaid multifunctional storage devices is not particularly limited in its usage, but its applications can be various such as a food storage container, a food aging container, a beverage storage container, a plastic bag, a mobile phone case, to name a few.

The aforesaid multi-functional powder may cause a beneficial effect to the human body based on far-infrared radiation feature. Specifically, the far-infrared rays by the multi-functional powder carries out a warming action maintaining the body temperature at an optimal level and a hydration/dehydration action keeping the human body at an appropriate level, and promotes metabolism while enhancing immunity through the balance of nutrients. In addition, it helps the expansion of capillaries and the cell tissue formation thereby promoting blood circulation and generating an effect of perspiration and pain relief, as well as enhanced metabolism.

In addition, the aforesaid multi-functional powder implements an effect to deodorize harmful gas, and in particular, has an excellent effect of deodorizing ammonia and formaldehyde, which are irritating and harmful to bio-tissues.

Furthermore, the multi-functional storage devices comprising the multi-functional powder have excellent capability of long-term storage and maintenance of a stored goods, as well as protection of the aforesaid stored goods from heavy metals contamination, microorganisms and harmful gases.

Presented in the following are specific embodiments of this Invention. However, the examples listed below are only intended to provide specific illustration or explanation of this Invention, which should not limit this Invention thereto.

### Implementation Examples and Comparative Examples

### Implementation Example 1

The multi-functional powder is manufactured, comprising the first component that contains calcium oxide (CaO), magnesium oxide (MgO) and aluminum oxide (Al2O3) by pulverizing refined natural mineral raw material into the size of 325 mesh, and the second component that contains sulfur trioxide (S3O) and strontium oxide (SrO). The composition is shown in Table 1 below. In Table 1, the weight ratio of the first component to the second component is 99.7:0.3, the weight ratio of magnesium oxide to aluminum oxide in the first component is 18.3:1, and the weight ratio of sulfur trioxide to strontium oxide in the second component is 5.4:1.

TABLE 1

| Component | | Weight % |
|---|---|---|
| The first component | calcium oxide | 94.0 |
| | magnesium oxide | 5.43 |
| | aluminum oxide | 0.296 |
| The second component | sulfur trioxide | 0.235 |
| | strontium oxide | 0.0432 |

Implementation Example 2 The multi-functional powder is manufactured, comprising the first component that contains silicon oxide (SiO2), aluminum oxide (Al2O3),calcium oxide (CaO), magnesium oxide (MgO), by pulverizing refined natural mineral raw material into the size of 325 mesh, and the second component that contains iron oxide (Fe2O3), sodium oxide (Na2O), boron oxide (B2O3), potassium oxide (K2O), titanium dioxide (TiO2), sulfur trioxide (S3O), manganese oxide (MnO), titanium dioxide (TiO2). The composition is shown in Table 2 below. In Table 2, the weight ratio of the first component to the second component is 97:3, and the total weight of the aforesaid silicon oxide and the aluminum oxide represent 97% in 100 weight% of the total first component, and the weight ratio of silicon oxide to the aluminum oxide is 1.09:1, and the weight ratio of the iron oxide to the sodium oxide is 1.43:1.

TABLE 2

| Component | | Weight % |
|---|---|---|
| The first component | silicon oxide | 49.44 |
| | aluminum oxide | 45.43 |
| | calcium oxide | 1.92 |
| | magnesium oxide | 0.31 |
| The second component | iron oxide | 1.01 |
| | sodium oxide | 0.71 |
| | boron oxide | 0.54 |
| | potassium oxide | 0.42 |
| | titanium dioxide | 0.14 |
| | sulfur trioxide | 0.06 |
| | manganese oxide | 0.02 |

### Evaluation

### Experiment Example 1: Evaluation of Far-Infrared Radiation Characteristics

For each of the multi-functional powders of aforesaid Implementation Examples 1 and 2, far-infrared radiation rate and radiant energy were measured by the KFIA-F1-1005 standard method. Specifically, it was measured at 37° C., and it is a measurement result against the black body using FT-1R Spectrometer. The results are as described in Table 3 below.

### Experiment Example 2: Evaluation of Harmful Gas Deodorization Performance

For each of the multi-functional powders in aforesaid Implementation Examples 1 and 2, a sample of 1.0 g was prepared, and ammonia gas or formaldehyde gas of 100 ppm volume % were injected in a tightly sealed space of 2 L volume under conditions of a temperature of 23±5° C. and a relative humidity of 45° C., followed by measuring the initial gas concentration value (Ci). Then, 1.0 g of the multi-functional powder is placed in the tightly sealed space and gas concentration value (Cf) is measured at each point of time when 30 minutes, 60 minutes, 90 minutes and 120 minutes have elapsed. Then, the ammonia deodorization rate and the formaldehyde deodorization rate were measured by the Formula 2 and 3 set out below, respectively, from which the harmful gas deodorization balance index of the following Formula 1 was derived. The results are as shown in Table 3 below.

$$\left\{\left(\text{Cai - Caf}\right) / \text{Cai}\right\} / \left\{\left(\text{Cfi - Cff}\right) / \text{Cfi}\right\} \qquad \text{[Formula 1]}$$

$$\text{Ammonia deodorization rate} \left(\%\right) = \left(\text{Cai - Caf}\right) \text{X 100} \qquad \text{[Formula 2]}$$

$$\text{Formaldehyde deodorization rate} \left(\%\right) = \left(\text{Cfi - Cff}\right) / \text{Cti} \text{X 100} \qquad \text{[Formula 3]}$$

In aforesaid Formulas 1 through 3, Cai is the initial gas concentration value after injecting 100 ppm volume % of ammonia gas into a tightly sealed space with the volume of 2 L under the temperature of 23±5° C. and a relative humidity of 45±10%; Caf is the ammonia gas concentration value after 120 minutes’ passage from placing 1.0 g of the multi-functional powder in the aforesaid tightly sealed space; Cfi is the initial gas concentration value after injecting 100 ppm volume% of formaldehyde gas into a tightly sealed space with the volume of 2 L under the temperature of 23±5° C. and a relative humidity of 45±10%; and Cff is the formaldehyde gas concentration value after 120 minutes’ passage from placing 1.0 g of the multi-functional powder in the aforesaid tightly sealed space.

### Experiment Example 3: Oxidation-Reduction Potential (ORP)

For each of the multifunctional powders of aforesaid Implementation Examples 1 and 2, a sample of 1.0 g was prepared and put into a conical tube, to which 10 mL of deionized water was added for 24-hour shaking, followed by extraction and centrifuging process to obtain a supernatant. ORP was measured for that supernatant. 1) For ORP measurement, water quality analyzer HORIBA LAQUA F-72 (Kyoto, Japan) was used. 2) For an electrode, HORIBA 9300-10DORP Electrode (Kyoto, Japan) was used. 3) Before measuring the ORP of the sample, ORP standard solution Quinhydrone 263 mv±30 mV, 25° C. (Thermo Scientific, Waltham MA, USA) was used, and a calibration process was completed. 4) After 10 times of measuring each sample, the mean and standard error were obtained. The results are shown in Table 3 below.

### Experiment Example 4: Hydrogen Ion Concentration (pH)

For each of the multi-functional powders in aforesaid Implementation Examples 1 and 2, a sample of 1.0 g was prepared, each of which was dispersed in 10 mL of distilled water. The resulting solution was placed at the temperature of 24±2° C. and a relative humidity of 35±5% for 30 minutes to produce a supernatant. The pH of the supernatant was measured using a Thermo/orion 5 star pH meter.

### Experiment Example 5: Cation Exchange Capacity (CEC) and Electrical Conductivity

For each of the multifunctional powders in aforesaid Implementation Examples 1 and 2, the cation exchange capacity (CEC) was measured by the following process. A device connected to the leaching tube and cleaning solution container and receptacle was installed. The leaching tube was composed of a leg with a length of 4 cm and an inner diameter of 0.3 cm and a tube with a length of 12 cm and an inner diameter of 1.3 cm. The container for the cleaning solution is of 100 ml capacity, with 10 ml markings. A small amount of cotton wool was inserted in the lower part of the aforesaid leaching tube to form a support layer, and filter sheets (6 types) are cut in small pieces and added thereon, put in water, boiled to make filter pulp, which is then made into filter plane with the thickness of 5 mm. Then, several ml’s of ammonium acetate solution is added to the lower part of the leaching tube, and 5 to 10 g of the multifunctional powder is added in small quantity to submerge, while adjusting the cork of the cleaning solution container to control the dropping rate so that 100 ml of ammonium acetate solution may penetrate within 4 to 8 hours. The penetrated solution is used for quantitative analysis of the exchangeable cation. After the penetration is complete, the inner upper part of the penetration tube is washed with a small quantity of alcohol, and the excess ammonium acetate solution is removed with 50 ml of alcohol. The analysis sample saturated with NH4+ ions are exchanged with 100 ml of a 10% sodium chloride solution for to substitute leach NH4+ for use as au non-treated solution. The whole or a certain quantity of the non-treated solution is placed in a distillation flask, and from the leachate, ammonia nitrogen is distilled and measured by nitrogen quantitative method. The figure obtained through quantifying NH4+ is converted to (me) per 100 g of sample, which is considered to be the cation exchange capacity.

For each of the multifunctional powders in aforesaid Implementation Examples 1 and 2, a sample of 10 g is taken, placed in a 100 ml Erlenmeyer flask, to which 50 ml of distilled water is added for 1 hour shake process. Then, it is filtered through a filter sheet and the electrical conductivity was measured using an electrical conductivity measuring device.

### Experiment Example 6: Average Particle Diameter

For each of the multifunctional powders in aforesaid Implementation Examples 1 and 2, mesh was used to implement sorting process, and the average particle diameter of each was calculated using an ordinary particle measuring analysis method.

### Experimental Example 7: Evaluation of Antibacterial Performance

For each of the multifunctional powders of aforesaid Implementation Examples 1 and 2, a sample of 3 g was prepared and mixed well with 100 mL of distilled water to prepare a mixed solution, followed by placing the mixed solution undisturbed for 5 minutes for precipitation, and the clear upper part of the solution was used for evaluation. Then the supernatant was treated for reaction with staphylococcus aureus (ATCC 6538), Escherichia coli (ATCC 8739), Klebsiella pneumonia (ATCC 4352), and Pseudomonas aeruginosa (ATCC 10145) for five minutes, respectively, followed by calculation of bacterial reduction rate (%) in accordance with Formula 4. The results are as indicated in Table 3 below.

$$\text{Bacterial reduction rate} \left(\%\right) = \left(\text{X-Y}\right) / \text{X} \text{*100} \qquad \text{[Formula 4]}$$

In Formula 4, aforesaid X is the number of colony-forming unit (CFU) in the controlled group, and Y is the number of CFU in the experimental group.

TABLE 3

| Evaluation result | | Unit | Implementation Example 1 | Implementation Example 2 |
|---|---|---|---|---|
| Far-infrared radiation characteristics | Radiation rate | % | 0.924 | 0.922 |
| | Radiation energy | W/m²•µm | 3.56 × 102 | 3.55 × 102 |
| Ammonia deodorization rate | 30 min. | % | 75 | 95 |
| | 60 min. | | 95 | 95 |
| | 90 min. | | 97 | 95 |
| | 120 min. | | 98 | 95 |
| Formaldehyde deodorization rate | 30 min. | % | 97 | 40 |
| | 60 min. | | 97 | 40 |
| | 90 min. | | 97 | 40 |
| | 120 min. | | 97 | 60 |
| Harmful gas deodorization balance index | | - | 1.01 | 1.58 |
| Oxidation-reduction potential | | mV | -8.56±0.81 | 186.87±11.75 |
| Hydrogen ion concentration | | - | 12.3 | 6.13 |
| Cation exchange capacity | | cmol(+)/kg | 43.80 | 17.70 |
| Electronic conductivity | | ds/m(1:10) | 8.52 | 0.08 |
| Average particle diameter | | µm(46) | 46±1 | 46±1 |
| Bacterial reduction rate | ATCC 6538 | % | 99.9 | - |
| | ATCC 8739 | | 99.9 | - |
| | ATCC 4352 | | 99.9 | - |
| | ATCC 10145 | | 99.9 | - |

Referring to Table 3, it can be confirmed that the multi-functional powders in the aforesaid Implementation Examples 1 and 2 have the advantage of excellent far-infrared radiation performance beneficial to the human body, and excellent effect of deodorizing ammonia and formaldehyde concurrently. Specifically, in aforesaid Implementation Examples 1 and 2, the multi-functional powder’s harmful gas deodorization balance index satisfies the range between 0.8 and 2.0, or, more specifically, between 0.9 and 1.7, or, more specifically, between 1.0 and 1.6 and thereby represent a corresponding deodorization balance with respect to the two harmful gases of ammonia and formaldehyde, which can be implemented with improved harmful gas removal capability in the application of the multifunctional powder (Figures are approximates except for identifiers.).

Notably, in the case of the multifunctional powder in aforesaid Implementation Example 1, the bacterial reduction rate for each of staphylococcus aureus (ATCC 6538), Escherichia coli (ATCC 8739), Klebsiella pneumonia (ATCC 4352), and Pseudomonas aeruginosa (ATCC 10145) exceeds 99%, thereby satisfying the range between 99.5% and 100%, for example, and as a result, a significant improvement of the function in antibacterial performance as well as in ammonia and formaldehyde deodorization performance is confirmed.

At the same time, the multifunctional powder in aforesaid Implementation Examples 1 and 2 satisfies predesignated ranges of oxidation reduction potential (ORP), hydrogen ion concentration (pH), cation exchange capacity (CEC) and electrical conductivity and as such can be utilized in various applications.

1. A multi-functional powder which: comprises the first component that includes one selection from the group consisting of silicon oxide (SiO2), aluminum oxide (Al2O3), calcium oxide (CaO), magnesium oxide (MgO), and combinations thereof; and comprises the second component that includes at least one selection from the group consisting of iron oxide (Fe2O3), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof; and whose far-infrared emission rate for the wavelength region of 5 µms to 20 µms at 37° C. is between 0.5% and 5%, and whose harmful gas deodorization balance index under the following Formula 1 is between 0.8 and 2.0.
$$\left\{\left(\text{Cai} \text{-} \text{Caf}\right) / \text{Cai}\right\} / \left\{\left(\text{Cfi} \text{-} \text{Cff}\right) / \text{Cfi}\right\} \qquad \text{[Formula 1]}$$
In Formula 1 above: Cai is the initial gas concentration value after injecting 100 ppm volume % of ammonia gas into a tightly sealed space with the volume of 2 L under the temperature of 23±5° C. and a relative humidity of 45±10%; Caf is the ammonia gas concentration value after 120 minutes’ passage from placing 1.0 g of the multi-functional powder in the aforesaid tightly sealed space; Cfi is the initial gas concentration value after injecting 100 ppm volume% of formaldehyde gas into a tightly sealed space with the volume of 2 L under the temperature of 23±5° C. and a relative humidity of 45±10%; and Cff is the formaldehyde gas concentration value after 120 minutes’ passage from placing 1.0 g of the multi-functional powder in the aforesaid tightly sealed space.

2. In relation to claim 1, a multifunctional powder with the ammonia deodorization rate of 90% or greater according to the following Formula 2.
$$\text{Ammonia} \text{deodorization} \text{rate} \left(\text{\%}\right) = \left(\text{Cai} \text{-} \text{Caf}\right) / \text{Cai} \times 100 \qquad \text{[Formula 2]}$$
In Formula 2, aforesaid Cai is the initial gas concentration value after injecting 100 ppm volume % of gas into a tightly sealed space with the volume of 2 L under the temperature of 23° C. and a relative humidity of 45%; and aforesaid Caf is the gas concentration value after 120 minutes’ passage from placing 1.0 g of the multi-functional powder in the aforesaid tightly sealed space.

3. In relation to claim 1, a multifunctional powder with a formaldehyde deodorization rate (%) of 50% or greater according to the following Formula 3:
$$\text{Formaldehyde} \text{deodorization} \text{rate} \left(\text{\%}\right) = \left(\text{Cfi} \text{-} \text{Cff}\right) / \text{Cfi} \times 100 \qquad \text{[Formula 3]}$$
In Formula 3, aforesaid Cfi is the initial gas concentration value after injecting 100 ppm volume % of gas into a tightly sealed space with the volume of 2 L under the temperature of 23° C. and a relative humidity of 45%; and aforesaid Cff is the gas concentration value after 120 minutes’ passage from placing 1.0 g of the multi-functional powder in the aforesaid tightly sealed space.

4. In relation to claim 1, a multi-functional powder with the bacterial reduction rate (%) of 90% or greater in accordance with the following Formula 4, for each of staphylococcus aureus (ATCC 6538), Escherichia coli (ATCC 8739), Klebsiella pneumonia (ATCC 4352), and Pseudomonas aeruginosa (ATCC 10145).
$$\text{Bacterial} \text{reduction} \text{rate} \left(\text{\%}\right) = \left(\text{X} \text{-} \text{Y}\right) / \text{X} \text{*} 100 \qquad \text{[Formula 4]}$$
In Formula 4, aforesaid X is the number of colony-forming unit (CFU) in the controlled group, and Y is the number of CFU in the experimental group treated with the multi-functional powder.

5. In relation to claim 1, a multifunctional powder with the weight ratio of its first component to its second component is 70:30 or greater and 100:0 or less.

6. In relation to claim 1, a multi-functional powder whose first component contains calcium oxide (CaO) and magnesium oxide (MgO) and whose second component contains sulfur trioxide (S3O) and strontium oxide (SrO), with the weight ratio of the first component to the second component being 95:5 or greater and 100:0 or less.

7. In relation to claim 6, a multi-functional powder which has calcium oxide representing 90 weight% or greater of the total 100 weight% of the aforesaid first component.

8. In relation to claim 1, a multi-functional powder whose first component contains silicon oxide (SiO2), aluminum oxide (Al2O3) and calcium oxide (CaO), and whose second component contains iron oxide (Fe2O3), with the weight ratio of the first component to the second component being 75:25 or greater and 100:0 or less.

9. In relation to claim 8, a multi-functional powder which has silicon oxide and aluminum oxide representing 80 weight% or greater of the total 100 weight% of the aforesaid first component.

10. A multi-functional storage device that contains functional material that comprises the multi-functional powder or a processed product thereof and a base material containing a resin or clay, as well as a storage target receiving part and a storage target protecting part and comprises the first component that includes one selection from the group consisting of silicon oxide (SiO2), aluminum oxide (Al2O3), calcium oxide (CaO), magnesium oxide (MgO), and combinations thereof; comprises the second component that includes at least one selection from the group consisting of iron oxide (Fe203), potassium oxide (K2O), sodium oxide (Na2O), strontium oxide (SrO), sulfur trioxide (S3O), boron oxide (B2O3), manganese oxide (MnO), titanium dioxide (TiO2), and combinations thereof; and whose far-infrared emission rate for the wavelength region of 5µms to 20µms at 37° C. is between 0.5% and 5%, and whose harmful gas deodorization balance index under the following Formula 1 is between 0.8 and 2.0.
$$\left\{\left(\text{Cai} \text{-} \text{Caf}\right) / \text{Cai}\right\} / \left\{\left(\text{Cfi} \text{-} \text{Cff}\right) / \text{Cfi}\right\} \qquad \text{[Formula 1]}$$
In Formula 1 above: Cai is the initial gas concentration value after injecting 100 ppm volume % of ammonia gas into a tightly sealed space with the volume of 2 L under the temperature of 23±5° C. and a relative humidity of 45±10%; Caf is the ammonia gas concentration value after 120 minutes’ passage from placing 1.0 g of the multi-functional powder in the aforesaid tightly sealed space; Cfi is the initial gas concentration value after injecting 100 ppm volume% of formaldehyde gas into a tightly sealed space with the volume of 2 L under the temperature of 23±5° C. and a relative humidity of 45±10%; and Cff is the formaldehyde gas concentration value after 120 minutes’ passage from placing 1.0 g of the multi-functional powder in the aforesaid tightly sealed space.
